(19) United States
(12) Patent Application Publication
(10) Pub. No.: US 2020/0015688 A1
(43) Pub. Date: Jan. 16, 2020

(54) BLOOD PRESSURE MEASUREMENT METHOD, DEVICE AND STORAGE MEDIUM

(71) Applicant: BOE TECHNOLOGY GROUP CO., LTD., Beijing (CN)

(72) Inventors: Guangfei LI, Beijing (CN); Xun ZHANG, Beijing (CN); Hui DU, Beijing (CN)

(73) Assignee: BOE TECHNOLOGY GROUP CO., LTD., Beijing (CN)

(21) Appl. No.: 16/507,441

(22) Filed: Jul. 10, 2019

(30) Foreign Application Priority Data
Jul. 10, 2018 (CN) 201810754761.4

(51) Int. Cl. A61B 5/021; A61B 5/00

(52) U.S. Cl. CPC A61B 5/02141; A61B 5/0077; A61B 5/7203; A61B 5/726; A61B 5/6816; A61B 5/6826; A61B 5/7257; A61B 5/725

(57) ABSTRACT

A blood pressure measurement method, a blood pressure measurement device, and a storage medium are provided. The method includes: acquiring a video of a part of a human body, and generating a PPGi signal based on the video; extracting feature information from the PPGi signal; fitting blood pressure models based on the feature information, and acquiring blood pressure data corresponding to each heartbeat, wherein the blood pressure models include a systolic blood pressure linear model and a diastolic blood pressure exponential model.

### CROSS-REFERENCE TO RELATED APPLICATION

This application claims priority to Chinese Patent Application No. 201810754761.4 filed on Jul. 10, 2018, which is incorporated herein by reference in its entirety.

### TECHNICAL FIELD

The present disclosure relates to the field of measurement, and more particularly relates to a blood pressure measurement method, a blood pressure measurement device, and a computer readable storage medium.

### BACKGROUND

Blood pressure refers to a lateral pressure applied on a blood vessel wall when blood flows in a blood vessel, and is one of basic indicators for evaluating a function of a cardiovascular system of a human body. There are two main types of blood pressure measurement methods. One of the two main types is a direct measurement method based on an arterial intubation method, and is frequently used for continuously monitoring a blood pressure of a patient during surgery. This method is invasive and has a high technical requirement and is not suitable for daily use. The other of the two main types is a cuff indirect measurement method, which is simple, accurate and non-invasive, but cannot continuously monitor the blood pressure.

### SUMMARY

The present disclosure provides a blood pressure measurement method, a blood pressure measurement device, and a nonvolatile computer readable storage medium.

In a first aspect, a blood pressure measurement method is provided in the present disclosure. The method includes: acquiring a video of a part of a human body, and generating a Photo-Plethysmography imaging (PPGi) signal P(t) based on the video; extracting feature information from the PPGi signal; fitting blood pressure models based on the feature information, and acquiring blood pressure data corresponding to each heartbeat, wherein the blood pressure models include a systolic blood pressure linear model and a diastolic blood pressure exponential model.

Optionally, the acquiring the video of the part of the human body, and generating the PPGi signal based on the video, includes: decomposing an image sequence in the video into image frames, and acquiring G-channel pixel information of each of the image frames; generating the PPGi signal based on the G-channel pixel information.

Optionally, the generating the PPGi signal based on the G-channel pixel information, includes: determining a value of the PPGi signal at a time point based on an average value of the G-channel pixel information of pixels in each of the image frames.

Optionally, the extracting the feature information from the PPGi signal, includes: acquiring feature information of each heartbeat based on the PPGi signal P(t), wherein the feature information includes a diastolic time (DT) and a waveform area parameter K, a formula for calculating the waveform area parameter K is as follows:

$$K = \frac{Pm - Pd}{Ps - Pd} ,$$

Ps, Pd, and Pm are respectively a maximum value, a minimum value, and an average value of the P(t) in a heartbeat period T, the average value Pm is calculated as follows:

$$Pm = \frac{1}{T} {\int }_{0}^{T} P ( t ) dt .$$

Optionally, the fitting the blood pressure models based on the feature information, and acquiring blood pressure data corresponding to each heartbeat, includes: fitting a systolic blood pressure (SBP) linear model based on the feature information, wherein the systolic blood pressure (SBP) linear model is as follows: SBP=a*DT+b wherein a and b are linear coefficients; fitting a diastolic blood pressure (DBP) exponential model based on the feature information, wherein the diastolic blood pressure (DBP) exponential model is as follows:

$$DBP = SBP * {e}^{\frac{DT}{c * K + d }} ,$$

wherein c and d are exponential coefficients.

Optionally, the fitting the blood pressure models based on the feature information, and acquiring blood pressure data corresponding to each heartbeat, further includes: acquiring a plurality of blood pressure data samples; substituting feature information of the plurality of blood pressure data samples into the systolic blood pressure linear model to determine values of a and b; substituting the feature information of the plurality of blood pressure data samples into the diastolic blood pressure exponential model to determine values of c and d.

Optionally, the systolic blood pressure (SBP) linear model is determined using the following formula: SBP−SBP₀=a*(DT−DT₀)+b, wherein, SBP₀ and DT₀ are calibrated values of a systolic blood pressure and a diastolic time.

Optionally, before the extracting the feature information from the PPGi signal, the blood pressure measurement method further includes: filtering and denoising the PPGi signal, wherein a bandpass filter is used to perform the filtering or a high-pass filter and a low-pass filter are combined to perform the filtering.

Optionally, the part of the human body is a fingertip or an earlobe of the human body.

Optionally, before the decomposing an image sequence in the video into image frames, the method further includes: converting a color mode of the video to a Red-Green-Blue (RGB) mode.

Optionally, the feature information includes a time-domain parameter, a frequency-domain parameter, a wavelet parameter, a morphological parameter, and a nonlinear parameter of the PPGi signal.

In a second aspect, a blood pressure measurement device is provided in the present disclosure. The blood pressure measurement device includes a processor, a storage connected to the processor, and at least one camera connected to the processor, wherein the storage stores programs and data, the processor is configured to read and execute the programs and data to control the at least one camera to acquire a video of a part of a human body; the processor is further configured to read and execute the programs and data to generate a Photo-Plethysmography imaging (PPGi) signal P(t) based on the video; extract feature information from the PPGi signal; fit blood pressure models based on the feature information, and acquire blood pressure data corresponding to each heartbeat, wherein the blood pressure models include a systolic blood pressure linear model and a diastolic blood pressure exponential model.

Optionally, the device further includes: a flashlight, wherein the processor is further configured to read and execute the programs and data to control the flashlight to emit light of a predetermined frequency.

In a third aspect, a blood pressure measurement device is provided in the present disclosure. The device includes: at least one camera, configured for acquiring a video of a body part; a Photo-Plethysmography imaging (PPGi) signal generation circuit, configured for acquiring the video from the at least one camera and generating a PPGi signal P(t) based on the video; a feature-information extraction circuit, configured for extracting feature information from the PPGi signal; a blood-model fitting circuit, configured for fitting blood pressure models based on the feature information, and acquiring blood pressure data corresponding to each heartbeat, wherein the blood pressure models include a systolic blood pressure linear model and a diastolic blood pressure exponential model.

Optionally, the PPGi signal generation circuit is specifically configured for: decomposing an image sequence in the video into image frames, and acquiring G-channel pixel information of each of the image frames; generating the PPGi signal based on the G-channel pixel information.

Optionally, the generating the PPGi signal based on the G-channel pixel information, includes: determining a value of the PPGi signal at a time point based on an average value of the G-channel pixel information of pixels in each of the image frames.

Optionally, the feature-information extraction circuit is specifically configured for: acquiring feature information of each heartbeat based on the PPGi signal P(t), wherein the feature information includes a diastolic time (DT) and a waveform area parameter K, a formula for calculating the waveform area parameter K is as follows:

$$K = \frac{Pm - Pd}{Ps - Pd} ,$$

Ps, Pd, and Pm are respectively a maximum value, a minimum value, and an average value of the P(t) in a heartbeat period T, the average value Pm is calculated as follows:

$$Pm = \frac{1}{T} {\int }_{0}^{T} P ( t ) dt .$$

Optionally, the blood-pressure-model fitting circuit is specifically configured for: fitting a systolic blood pressure (SBP) linear model based on the feature information, wherein the systolic blood pressure (SBP) linear model is as follows: SBP=a*DT+b, wherein a and b are linear coefficients; fitting a diastolic blood pressure (DBP) exponential model based on the feature information, wherein the diastolic blood pressure (DBP) exponential model is as follows:

$$DBP = SBP * {e}^{\frac{DT}{c * K + d}} ,$$

wherein c and d are exponential coefficients.

Optionally, the blood-pressure-model fitting circuit is further configured for: acquiring a plurality of blood pressure data samples; substituting feature information of the plurality of blood pressure data samples into the systolic blood pressure linear model to determine values of a and b; substituting the feature information of the plurality of blood pressure data samples into the diastolic blood pressure exponential model to determine values of c and d.

In a third aspect, a non-volatile computer readable storage medium is provided in the present disclosure. The storage medium includes computer programs stored on the non-volatile computer readable storage medium, wherein when the computer programs are executed by a processor, the processor implements the blood pressure measurement method according to the first aspect.

### BRIEF DESCRIPTION OF THE DRAWINGS

Other features, objects, and advantages of the present disclosure will become more apparent by reading a detailed description of the embodiments with reference to the drawings.

FIG. 1 illustrates an exemplary flowchart of a blood pressure measurement method according to embodiments of the present disclosure;

FIG. 2 illustrates an exemplary flowchart of a step S10 of the blood pressure measurement method according to the embodiments of the present disclosure;

FIG. 3 illustrates an exemplary flow chart of a substep S12 of the step S10 according to the embodiments of the present disclosure;

FIG. 4 illustrates an exemplary schematic diagram of a PPGi signal according to the embodiments of the present disclosure;

FIG. 5 illustrates an exemplary flowchart of a step S20 of the blood pressure measurement method according to the embodiments of the present disclosure;

FIG. 6 illustrates an exemplary flowchart of a step S30 of the blood pressure measurement method according to the embodiments of the present disclosure;

FIG. 7 illustrates an exemplary flow chart of determining coefficients according to the embodiments of the present disclosure;

FIG. 8 illustrates another exemplary schematic diagram of the blood pressure measurement method according to the embodiments of the present disclosure;

FIG. 9 illustrates an exemplary structural block diagram of a blood pressure measurement device according to the embodiments of the present disclosure;

FIG. 10 illustrates another exemplary structural block diagram of the blood pressure measurement device according to the present disclosure.

### DETAILED DESCRIPTION

The present disclosure will be further described in details below in conjunction with accompanying drawings and embodiments. It is understood that the specific embodiments described herein are merely illustrative of the present disclosure, rather than used to limit the present disclosure. It should also be noted that, for convenience of description, only parts related to the present disclosure are shown in the drawings.

It should be noted that the embodiments in the present disclosure and the features in the embodiments may be combined with each other without conflicting with each other. The present disclosure will be described in details below with reference to the drawings and the embodiments.

A blood pressure of a person, especially middle-aged or older patients and high-pressure patients, may easily and substantially change with changes of the environment and a mood of the person. If the blood pressure changes rapidly in a short period of time, a great damage may be introduced to a cardiovascular system of the person, and possibly a life-threatening may be brought to the person when a condition of the blood pressure is serious. Therefore, a cuff method has limitations in measuring the blood pressure, and a continuous blood pressure monitoring method is needed, which is simple to be operated, superior in performance, and may measure a value of the blood pressure in real time and record and store blood pressure data.

Referring to FIG. 1, FIG. 1 shows an exemplary flow chart of a blood pressure measurement method according to embodiments of the present disclosure. As shown in FIG. 1, the blood pressure measurement method includes steps S10-S30.

Step S10: acquiring a video of a part of a human body, and generating a Photo-Plethysmography imaging (PPGi) signal based on the video of the part of the human body.

Step S20: extracting feature information from the PPGi signal.

Step S30: fitting blood pressure models based on the feature information, and acquiring blood pressure data corresponding to each heartbeat, wherein the blood pressure models include a systolic blood pressure linear model and a diastolic blood pressure exponential model.

In a specific application, the part of a human body may be a fingertip or an earlobe of the human body. A video of the fingertip or the earlobe of the human body may be acquired by a camera, to generate the Photo-Plethysmography imaging (PPGi) signal based on the video. The PPGi signal is a signal generated based on a Photo-Plethysmography imaging technology. The PPGi technology uses a camera to acquire a Photoplethysmographic signal and acquires features of a human body based on the Photoplethysmographic signal.

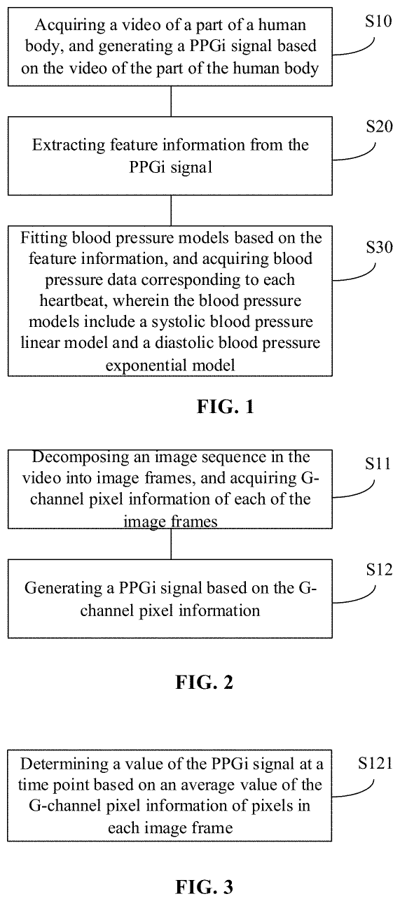

Referring to FIG. 2, FIG. 2 shows an exemplary flowchart of a step S10 of the blood pressure measurement method according to the embodiments of the present disclosure. As shown in FIG. 2, the step S10 includes the following substeps S11-S12.

Substep S11: decomposing an image sequence in the video into image frames, and acquiring G-channel pixel information of each of the image frames.

Substep S12: generating a PPGi signal based on the G-channel pixel information.

Specifically, a color mode of the video acquired originally may be an RGB (Red, Green, Blue) mode. If the color mode of the video is another mode such as a HSL (hue, saturation, lightness) mode, a HSV (hue, saturation, value) mode, and the like, the color mode needs to be converted to the RGB mode. Thereafter, the acquired video is decomposed into a sequence of image frames, and each of the image frames is decomposed into pixel information of three channels R, G, and B. In view of a high light absorption of hemoglobin in a green spectral range, the PPGi signal is generated by using the G-channel pixel information.

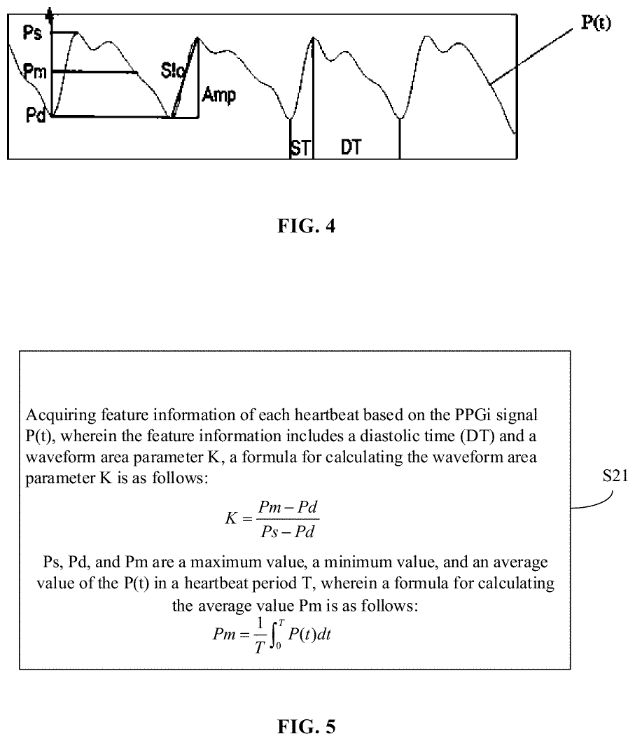

Next, referring to FIGS. 3-4, FIG. 3 shows an exemplary flowchart of a substep S12 according to the embodiments of the present disclosure; FIG. 4 shows an exemplary schematic diagram of a PPGi signal according to the embodiments of the present disclosure. As shown in FIG. 3, the substep S12 includes a sub-substep S121.

Sub-substep S121: determining a value of the PPGi signal at a time point based on an average value of the G-channel pixel information of pixels in each image frame.

Specifically, when a frame rate of the camera is 30 fps, that is, 30 images are taken within 1 second, the PPGi signal generated corresponding to the 30 images has 30 values within 1 second, and a sampling rate for the PPGi signal is considered to be 30 Hz. FIG. 4 shows an example of the PPGi signal.

Referring to FIG. 5, FIG. 5 illustrates an exemplary flowchart of the step S20 of the blood pressure measurement method according to the embodiments of the present disclosure. As shown in FIG. 5, the step S20 includes a substep S21.

Substep S21: acquiring feature information of each heartbeat based on the PPGi signal P(t), wherein the feature information includes a diastolic time (DT) and a waveform area parameter K, a formula for calculating the waveform area parameter K is as follows:

$$K = \frac{Pm - Pd}{Ps - Pd} , .$$

Ps, Pd, and Pm are a maximum value, a minimum value, and an average value of the P(t) in a heartbeat period T, wherein a formula for calculating the average value Pm is as follows:

$$Pm = \frac{1}{T} {\int }_{0}^{T} P ( t ) dt .$$

The feature information may be extracted from the PPGi signal. The feature information may be selected from, but is not limited to, following types: a time-domain parameter, a frequency-domain parameter, a wavelet parameter, a morphological parameter, a nonlinear parameter of the PPGi signal, and the like.

Taking the time-domain parameter as an example, a systolic time (ST) from a trough to a crest of a pulse wave of each heartbeat, a diastolic time (DT) from a crest to a trough, a waveform amplitude height Amp, and an ascending slope Slo from the trough to the crest are extracted respectively. The present disclosure mainly extracts the diastolic time (DT) and the waveform area parameter K.

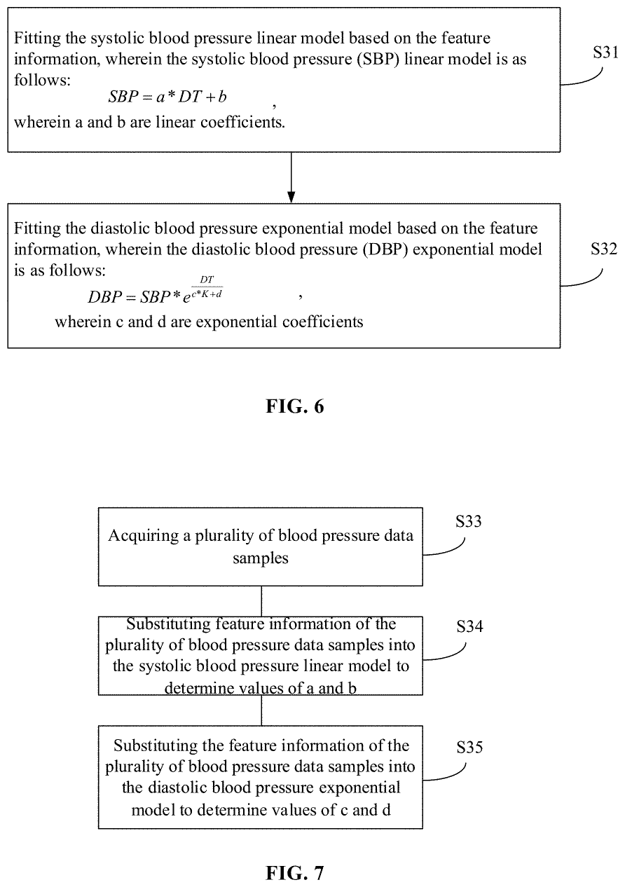

Referring to FIG. 6, FIG. 6 shows an exemplary flowchart of the step S30 of the blood pressure measurement method according to the embodiments of the present disclosure. As shown in FIG. 6, the step S30 includes substeps S31-S32.

Substep S31: fitting the systolic blood pressure linear model based on the feature information, wherein the systolic blood pressure (SBP) linear model is as follows:

SBP=a*DT+b

wherein a and b are linear coefficients.

Substep S32: fitting the diastolic blood pressure exponential model based on the feature information, wherein the diastolic blood pressure (DBP) exponential model is as follows:

$$DBP = SBP * {e}^{\frac{DT}{c * K + d}} ,$$

wherein c and d are exponential coefficients.

A method for fitting the blood pressure models may be selected from, but is not limited to, the following categories: a least-square method, a ridge regression method, a tree regression method, a support vector machine, a similarity matching method, and the like. The present disclosure fits the systolic blood pressure SBP using a linear least-square method, and fits the diastolic blood pressure (DBP) using the exponential model.

Next, referring to FIG. 7, FIG. 7 illustrates an exemplary flowchart of a manner for determining the above coefficients according to the embodiments of the present disclosure. The method for determining the coefficients a, b, c, d includes steps S33-S35.

Step S33: acquiring a plurality of blood pressure data samples.

Step S34: substituting feature information of the plurality of blood pressure data samples into the systolic blood pressure linear model to determine values of a and b.

Step S35: substituting the feature information of the plurality of blood pressure data samples into the diastolic blood pressure exponential model to determine values of c and d.

Specifically, values of the systolic blood pressure and the diastolic time of the samples are substituted into the systolic blood pressure linear model to determine the value of a and the value of b. The value of the systolic blood pressure, the value of the diastolic blood pressure, the diastolic time, and the K of the samples are substituted into the diastolic blood pressure exponential model to determine the value of c and the value of d.

In some embodiments, the systolic blood pressure (SBP) linear model may also be determined using the following formula:

SBP−SBP₀=a*(DT−DT₀)+b,

wherein, SBP₀ and DT₀ are calibrated values of the systolic blood pressure and the diastolic time. This method may reduce differences among individuals and improve an accuracy of the measurement. During use, the SBP₀ and the DT₀ of the measured object are substituted into the above formula for performing the calculation.

In some embodiments, before extracting the feature information from the PPGi signal, the blood pressure measurement method further includes filtering and denoising the PPGi signal, wherein a bandpass filter is used for filtering.

In some embodiments, prior to extracting the feature information from the PPGi signal, the blood pressure measurement method further includes filtering and denoising the PPGi signal, wherein a high-pass filter and a low-pass filter are combined to perform the filtering.

Frequencies of the PPGi signal are mainly concentrated between 0.1-30 Hz. A frame rate of the camera is generally in an order of tens of fps. For example, a rear camera on an ordinary smartphone has a frame rate of 30 fps, and a corresponding sampling rate of the PPGi signal is 30 Hz. When a passband of the bandpass filter is 0.6-4.5 Hz, high-frequency interferences, glitches and low-frequency baseline drifts may be significantly removed.

For the passband, a finite impulse response (FIR) filter may be directly used to perform the filtering, such as a 0.6-4.5 Hz bandpass filter or a combination of a 4.5 Hz lowpass filter and a 0.6 Hz highpass filter. Since a normalized passband is 0.04-0.3 (the passband 0.6-4.5 Hz divided by a half of the sampling rate of 15 Hz), causing a left falling edge of the bandpass filter to be too steep and an order of the filter is too large. Thus, a 4.5 Hz low-pass filter and a 0.6 Hz high-pass filter are combined to filter out noise. The above only gives a method of filtering and denoising, and the method of filtering and denoising may be adjusted according to situations in actual applications.

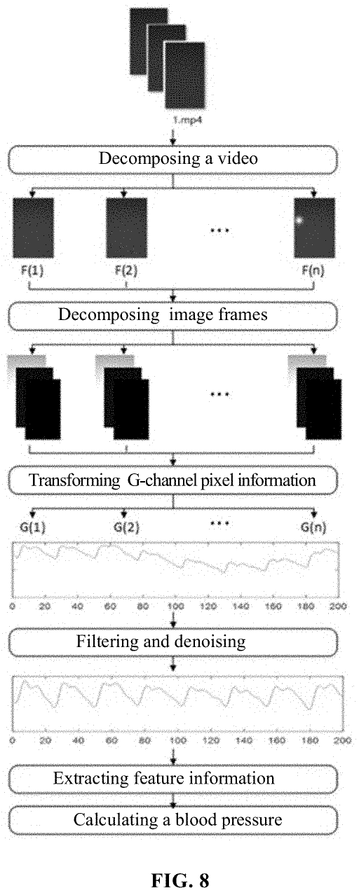

FIG. 8 illustrates another exemplary schematic diagram of a blood pressure measurement method according to the embodiments of the present disclosure. The blood pressure measurement method as shown in FIG. 8 includes: decomposing the acquired video into a sequence of image frames; decomposing each image frame into pixel information of a Red (R) channel, a Green (G) channel and a Blue (B) channel, and extracting an average value of the G-channel pixel information as an original PPGi signal; filtering and denoising the original PPGi signal, and extracting feature information; fitting blood pressure measurement models based on the feature information to measure the blood pressure.

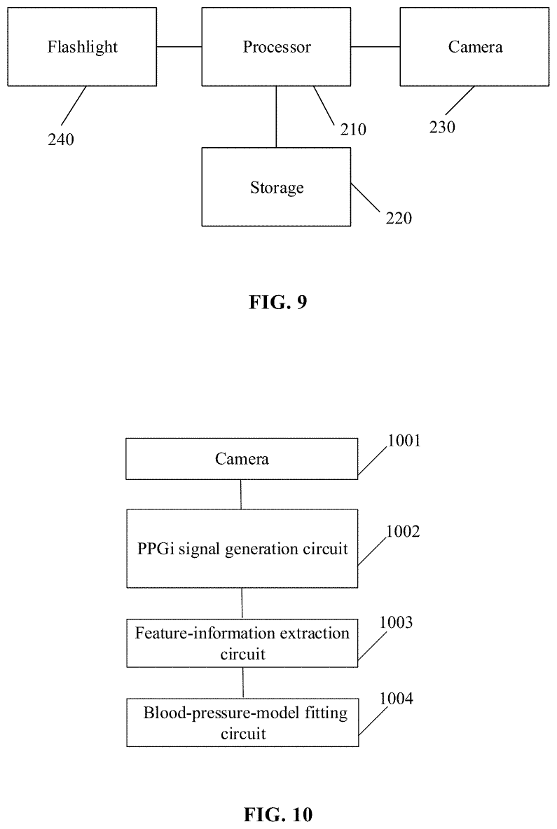

FIG. 9 shows an exemplary structural block diagram of a blood pressure measurement device according to the embodiments of the present disclosure. The device shown in FIG. 9 includes a processor 210, a storage 220 connected to the processor 210, and at least one camera 230 connected to the processor 210. The storage 220 stores programs and data. The processor 210 is configured to read and execute the programs and the data stored in the storage 220 to control the at least one camera 220 to acquire a video of a part of a human body.

It should be noted that a light source required during acquiring the video may be an external natural light source, or may be a Light Emitting Diode (LED) light source such as a flashlight in a mobile phone or a near-infrared LED.

Optionally, the device further includes a flashlight 240. The processor 210 is further configured to read and execute programs and data stored in the storage 220 to control the flashlight 240 to emit light having a predetermined frequency so as to assist in acquiring the video of the part of the human body.

The processor 210 is further configured to read and execute the programs and the data stored in the storage 220, to generate a PPGi signal based on the acquired video, extract feature information, fit blood pressure models, and acquire blood pressure data corresponding to each heartbeat, wherein the blood pressure models include a systolic blood pressure linear model and a diastolic blood pressure exponential mode.

FIG. 10 illustrates another exemplary structural block diagram of a device according to the embodiments of the present disclosure. The device illustrated in FIG. 10 includes at least one camera 1001, a PPGi signal generation circuit 1002, a feature-information extraction circuit 1003, and a blood-pressure-model fitting circuit 1004. The at least one camera 1001 is configured to acquire a video of a part of a human body. The PPGi signal generation circuit 1002 is configured to acquire the video from the at least one camera 1001 and generate a Photo-Plethysmography imaging (PPGi) signal based on the video.

The feature-information extraction circuit 1003 is configured to extract feature information from the PPGi signal. The blood-pressure-model fitting circuit 1005 is configured to fit blood pressure models based on the feature information, and acquire blood pressure data corresponding to each heartbeat, wherein the blood pressure models include a systolic blood pressure linear model and a diastolic blood pressure exponential model.

Optionally, the PPGi-signal generation circuit 1002 is specifically configured to: decompose an image sequence in the video into image frames, acquire G-channel pixel information of the image frames; and generate the PPGi signal based on the G-channel pixel information.

The step of generating, based on the G-channel pixel information, the PPGi signal includes: determining a value of the PPGi signal at a time point based on an average value of the G-channel pixel information of pixels in each image.

Optionally, the feature information extraction circuit 1003 is configured to: acquire feature information of each heartbeat based on the PPGi signal P(t), wherein the feature information includes a diastolic time (DT) and a waveform area parameter K, a formula for calculating the waveform area parameter K is as follows:

$$K = \frac{Pm - Pd}{Ps - Pd}$$

Ps, Pd, and Pm are a maximum value, a minimum value, and an average value of the P(t) in a heartbeat period T, wherein a formula for calculating the average value Pm is as follows:

$$Pm = \frac{1}{T} {\int }_{0}^{T} P ( t ) dt .$$

Optionally, the blood-pressure-model fitting circuit 1004 is specifically configured to fit the systolic blood pressure linear model based on the feature information, wherein the systolic blood pressure (SBP) linear model is as follows:

SBP=a*DT+b,

wherein a and b are linear coefficients.

The blood-pressure-model fitting circuit 1004 is specifically configured to fit the diastolic blood pressure (DBP) exponential model based on the feature information, wherein the diastolic blood pressure (DBP) exponential model is as follows:

$$DBP = SBP * {e}^{\frac{DT}{c * K + d}} ,$$

wherein c and d are exponential coefficients.

The blood-pressure-model fitting circuit is further configured to: acquire a plurality of blood pressure data samples; substitute feature information of the plurality of blood pressure data samples into the systolic blood pressure linear model to determine a and b; and substitute the feature information of the plurality of blood pressure data samples into the diastolic blood pressure exponential model to determine c and d.

It should be noted that a light source required for capturing the video may be an external natural light source, or may adopt a Light Emitting Diode (LED) light source such as a flashlight in a mobile phone or a near-infrared light LED.

The blood pressure measurement device provided by the present disclosure is a device corresponding to the blood pressure measurement method provided by the present disclosure, and may implement all the technical contents of the blood pressure measurement method of the present disclosure. Therefore, the content of the blood pressure measurement device of the present disclosure may be acquired by referring to the above blood pressure measurement method, and will not be described in detail herein.

According to the technical solutions provided by the embodiments of the present disclosure, the PPGi signal is generated by acquiring the video of the fingertip or the earlobe of the human body through the camera; and the blood pressure data of each heartbeat is acquired based on the PPGi signal. The solutions of the present disclosure may solve the problem that a relevant blood pressure measurement method has an issue of not being adaptable to a continuous measurement.

Flowcharts and block diagrams in the drawings illustrate architecture, functionality, and operation of possible implementations of systems, methods and computer program products according to various embodiments of the invention. In this regard, each block of the flowcharts or block diagrams may represent a module, a program segment, or a portion of codes, the module, the program segment or the portion of codes include one or more executable instructions for implementing specified logical functions. It should also be noted that in some implementations, the functions noted in the blocks may also occur in an order different from that illustrated in the drawings. For example, depending upon functionality involved by two successively represented blocks, the two successively represented blocks may in fact be executed substantially in parallel, or may sometimes be executed in an order reverse to that shown in the drawings. It is also noted that each block of the block diagrams and/or flowcharts, and combinations of blocks in the block diagrams and/or flowcharts, may be implemented in a dedicated hardware-based system that performs a specified function or operation, or may be implemented by a combination of dedicated hardware and computer instructions.

In another aspect, the present disclosure further provides a computer readable storage medium. The computer readable storage medium may be a computer readable storage medium included in the device described in the foregoing embodiments, or may a computer readable storage medium existing independently and not assembled into the device. The computer readable storage medium stores programs and data. When the programs and data are executed by a processor, the processor performs the blood pressure measurement methods of the present disclosure. The computer readable storage medium may be a volatile computer readable storage medium or a non-volatile computer readable storage medium, such as a Read-Only Memory (ROM), a Random Access Memory (RAM), a magnetic disk or a compact disc (CD), or the like.

The above description is only illustrative of optional embodiments of the present disclosure and is an illustration of the principles of the applied technology of the present disclosure. It should be understood by those skilled in the art that the scope of the present disclosure is not limited to a technical solution formed by a specific combination of the above technical features, and without departing from the inventive concept of the present disclosure, should also be covered by other technical solutions formed by any combination of the above technical features and equivalent features, such as technical solutions formed by substituting the technical features with technical features disclosed in the present disclosure (not limited to the technical features disclosed in the present disclosure) and having similar functions.

What is claimed is:

1. A blood pressure measurement method comprising:
acquiring a video of a part of a human body, and generating a Photo-Plethysmography imaging (PPGi) signal P(t) based on the video;
extracting feature information from the PPGi signal;
fitting blood pressure models based on the feature information; and
acquiring blood pressure data corresponding to each heartbeat;
wherein the blood pressure models comprise a systolic blood pressure linear model and a diastolic blood pressure exponential model.

2. The blood pressure measurement method according to claim 1, wherein the acquiring the video of the part of the human body and generating the PPGi signal based on the video comprises:
decomposing an image sequence in the video into image frames, and acquiring G-channel pixel information of each of the image frames;
generating the PPGi signal based on the G-channel pixel information.

3. The blood pressure measurement method according to claim 2, wherein the generating the PPGi signal based on the G-channel pixel information comprises:
determining a value of the PPGi signal at a time point based on an average value of the G-channel pixel information of pixels in each of the image frames.

4. The blood pressure measurement method according to claim 1, wherein the extracting the feature information from the PPGi signal comprises:
acquiring feature information of each heartbeat based on the PPGi signal P(t), wherein the feature information comprises a diastolic time (DT) and a waveform area parameter K, a formula for calculating the waveform area parameter K is as follows:
$$K = \frac{Pm - Pd}{Ps - Pd} ,$$
Ps, Pd, and Pm are respectively a maximum value, a minimum value, and an average value of the P(t) in a heartbeat period T, the average value Pm is calculated as follows:
$$Pm = \frac{1}{T} {\int }_{0}^{T} P ( t ) dt .$$

5. The blood pressure measurement method according to claim 1, wherein the fitting the blood pressure models based on the feature information and acquiring blood pressure data corresponding to each heartbeat comprises:
fitting a systolic blood pressure (SBP) linear model based on the feature information, wherein the systolic blood pressure (SBP) linear model is as follows:
SBP=a*DT+b, wherein a and b are linear coefficients;
fitting a diastolic blood pressure (DBP) exponential model based on the feature information, wherein the diastolic blood pressure (DBP) exponential model is as follows:
$$DBP = SBP * {e}^{\frac{DT}{c * K + d}} ,$$
wherein c and d are exponential coefficients.

6. The blood pressure measurement method according to claim 5, wherein the fitting the blood pressure models based on the feature information and acquiring blood pressure data corresponding to each heartbeat further comprises:
acquiring a plurality of blood pressure data samples;
substituting feature information of the plurality of blood pressure data samples into the systolic blood pressure linear model to determine values of a and b;
substituting the feature information of the plurality of blood pressure data samples into the diastolic blood pressure exponential model to determine values of c and d.

7. The blood pressure measurement method according to claim 5, wherein the systolic blood pressure (SBP) linear model is determined using the following formula:
SBP−SBP₀=ₐ*(DT−DT₀)+b,
wherein, SBP₀ and DT₀ are calibrated values of a systolic blood pressure and a diastolic time.

8. The blood pressure measurement method according to claim 3, wherein before the extracting the feature information from the PPGi signal, the blood pressure measurement method further comprises:
filtering and denoising the PPGi signal, wherein a bandpass filter is used to perform the filtering or a high-pass filter and a low-pass filter are combined to perform the filtering.

9. The blood pressure measurement method according to claim 1, wherein the part of the human body is a fingertip or an earlobe of the human body.

10. The blood pressure measurement method according to claim 2, wherein before the decomposing an image sequence in the video into image frames, the method further comprises:
converting a color mode of the video to a Red-Green-Blue (RGB) mode.

11. The blood pressure measurement method according to claim 1, wherein the feature information comprises a time-domain parameter, a frequency-domain parameter, a wavelet parameter, a morphological parameter, and a nonlinear parameter of the PPGi signal.

12. A blood pressure measurement device comprising:
a processor, a storage connected to the processor for storing programs and data, and at least one camera connected to the processor,
wherein the processor is configured to read and execute the programs and data to control the at least one camera to acquire a video of a part of a human body, and the processor is further configured to read and execute the programs and data to generate a Photo-Plethysmography imaging (PPGi) signal P(t) based on the video, extract feature information from the PPGi signal, fit blood pressure models based on the feature information, and acquire blood pressure data corresponding to each heartbeat, wherein the blood pressure models comprise a systolic blood pressure linear model and a diastolic blood pressure exponential model.

13. The device according to claim 12 further comprising:
a flashlight, wherein the processor is further configured to read and execute the programs and data to control the flashlight to emit light of a predetermined frequency.

14. A blood pressure measurement device comprising:
at least one camera configured for acquiring a video of a body part;
a Photo-Plethysmography imaging (PPGi) signal generation circuit configured for acquiring the video from the at least one camera and generating a PPGi signal P(t) based on the video;
a feature-information extraction circuit configured for extracting feature information from the PPGi signal; and
a blood-model fitting circuit configured for fitting blood pressure models based on the feature information, and acquiring blood pressure data corresponding to each heartbeat, wherein the blood pressure models comprise a systolic blood pressure linear model and a diastolic blood pressure exponential model.

15. The device according to claim 14, wherein the PPGi signal generation circuit is specifically configured for:
decomposing an image sequence in the video into image frames, and acquiring G-channel pixel information of each of the image frames;
generating the PPGi signal based on the G-channel pixel information.

16. The device according to claim 15, wherein the generating the PPGi signal based on the G-channel pixel information comprises:
determining a value of the PPGi signal at a time point based on an average value of the G-channel pixel information of pixels in each of the image frames.

17. The device according to claim 14, wherein the feature-information extraction circuit is specifically configured for:
acquiring feature information of each heartbeat based on the PPGi signal P(t), wherein the feature information comprises a diastolic time (DT) and a waveform area parameter K, wherein a formula for calculating the waveform area parameter K is as follows:
$$K = \frac{Pm - Pd}{Ps - Pd} ,$$
wherein Ps, Pd, and Pm are respectively a maximum value, a minimum value, and an average value of the P(t) in a heartbeat period T, and the average value Pm is calculated as follows:
$$Pm = \frac{1}{T} {\int }_{0}^{T} P ( t ) dt .$$

18. The device according to claim 14, wherein the blood-pressure-model fitting circuit is specifically configured for:
fitting a systolic blood pressure (SBP) linear model based on the feature information, wherein the systolic blood pressure (SBP) linear model is as follows:
SBP=a*DT+b, wherein a and b are linear coefficients;
fitting a diastolic blood pressure (DBP) exponential model based on the feature information, wherein the diastolic blood pressure (DBP) exponential model is as follows:
$$DBP = SBP * {e}^{\frac{DT}{c * K + d}} ,$$
wherein c and d are exponential coefficients.

19. The device according to claim 18, wherein the blood-pressure-model fitting circuit is further configured for:
acquiring a plurality of blood pressure data samples;
substituting feature information of the plurality of blood pressure data samples into the systolic blood pressure linear model to determine values of a and b; and
substituting the feature information of the plurality of blood pressure data samples into the diastolic blood pressure exponential model to determine values of c and d.

20. A non-volatile computer readable storage medium comprising:
computer programs stored on the non-volatile computer readable storage medium, wherein when the computer programs are executed by a processor, the processor implements the blood pressure measurement method according to claim 1.
